(19) United States
(12) Patent Application Publication
(10) Pub. No.: US 2007/0122872 A1
(43) Pub. Date: May 31, 2007

(54) Method for storage of clinical samples prior to culture

(76) Inventors: Thomas Anthony Cumbo, Buffalo, NY (US); Timothy F. Murphy, East Amherst, NY (US)

Correspondence Address: HODGSON RUSS LLP, ONE M & T PLAZA, SUITE 2000, BUFFALO, NY, 14203-2391, US

(21) Appl. No.: 11/581,550

(22) Filed: Oct. 16, 2006

Related U.S. Application Data
(60) Provisional application No. 60/726,667, filed on Oct. 14, 2005.

(51) Int. Cl. C12Q 1/04; C12M 3/00

(52) U.S. Cl. 435/34; 435/287.1

(57) ABSTRACT

The present invention provides a storage media for the storage and transport of clinical samples. The storage medium comprises a microbiological growth medium, glycerol and a reducing agent. Clinical samples, such as sputum, can be added to the storage medium at the point of collection and then transported in the storage medium at ambient temperatures, refrigerated or as frozen samples. The yield of pathogens using the storage medium is significantly improved. Also provided is a device for the storage and transport of clinical samples which can be used in conjunction with the storage media described herein.

This application claims priority to U.S. provisional application No. 60/726,667, filed on Oct. 14, 2005, the disclosure of which is incorporated herein by reference.

### FIELD OF THE INVENTION

The present invention relates generally to the field of storage and transport of clinical samples.

### DISCUSSION OF RELATED ART

Over the past several years, financial and regulatory pressures have encouraged many health care delivery systems to centralize laboratory facilities. Prior to this ubiquitous change, microbiologic culture of diagnostic specimens was performed in laboratories located within the institution where the specimen was collected. As such, collection and transport of the specimen was more manageable and was performed in a timely manner. More recently, one centralized laboratory facility may serve multiple hospital and outpatient clinics with periodic transfer of specimens occurring in somewhat erratic fashion. The effect of such transfer on subsequent microbiologic yield and integrity has not been adequately evaluated but is a pressing concern to clinical laboratories and is of special concern when evaluating samples (such as sputum and stool) that potentially contain both pathologic bacteria and non-pathologic resident flora. The rapid growth of certain species at room temperature may eliminate other species. Despite the almost inevitable progression into more centralized national and international facilities, concerns remain about proper yield of relevant pathogens.

Among the various clinical tests, sputum culture is one of the most commonly used procedure. Health care practitioners routinely order sputum cultures from a variety of patients. These include inpatients who are critically ill, nursing home patients and outpatients. Sputum is viscous and compartmentalized. A standard practice in sputum culture is to obtain a sample by placing a wire loop in one area of a given sample. However, such a procedure may miss pathogenic bacteria “trapped” in a separate area, as has been demonstrated previously with such important pathogens as P. aeruginosa, S. pneumoniae, H. influenzae and K. pneumoniae. There are no currently acceptable methods for placing sputum samples in “suspended animation” after collection and prior to culture. In many instances, culture of the sample is delayed for hours or even days during which time medically important pathogens may be killed or overgrown with other less clinically relevant species of bacteria or fungi. Additionally, there is no currently acceptable method for transporting sputum samples to a centralized location for multi-center clinical trials or for international research studies.

Another biological sample used for diagnostic testing is stool. Stool samples are routinely collected for diagnostic and research purposes worldwide. In developed countries stool samples are routinely examined in patients with pre-existing conditions or epidemiological exposure to potentially pathologic organisms such as those with AIDS or international travelers. Further, in the developing countries, stool samples are an inexpensive way to aid the diagnosis of a myriad of protozoan and bacterial diseases. Finally, multiple research protocols in many areas of the world utilize stool samples for a variety of research.

Rapid loss of specimen integrity also presents a mammoth impediment to both international infectious diseases research collaboration as well as study of pathogens in remote areas. Therefore, it remains impossible to reliably collect a diagnostic or research specimen from a remote location. In the few locations where culture can be performed in the field, expensive technological and practical barriers exist. It is also virtually impossible to transfer a diagnostic specimen to a more specialized laboratory and ensure diagnostic accuracy; this is especially relevant given the potential for delay and temperature fluctuation during international shipping. There are no established protocols for preserving such specimens. Recovery and detection of bacteria such as Acinetobacter baumannii, Pseudomonas aeruginosa, Stenotrophomonas maltophilia, extended-spectrum beta-lactamase producing Enterobacteraciae, methicillin-resistant Staphylococcus aureus, Hemophilus influenzae and multi-drug resistant erm/mef+Streptococcus pneumoniae as well as Helicobacter pylori is critical for diagnosis and therapeutic measures.

Accordingly, there is a need in the area of clinical diagnostics for developing a reliable method for the storage and transport of sputum and other biological samples such that the pathogens can be identified even after significant storage and transport. A list of general references in this field are provided following the Examples.

### SUMMARY OF THE INVENTION

The present invention provides compositions and methods for storage and transportation of biological samples. Examples of such samples include, but are not limited to, sputum, stool, gastric and intestinal contents, and the like. The composition (referred to herein as the “storage medium” or “media” comprises a microbiological growth medium, 15-45% glycerol and a reducing agent. Biological samples can be stored in the storage composition as described herein. The use of the present composition improves preservation and retrieval of significant pathogens in the biological samples. The improvement may be in the form of increased number of pathogens being detected, increased numbers quantity of each or increased consistency of detection. The samples thus prepared can be stored at ambient temperatures for up to 24 hours, at refrigerated temperatures for up to 7 days and at freezer temperature (−20° C.) for at least 3 months.

The present invention also provides a device for storage and transport of the biological samples using the composition of the invention.

### BRIEF DESCRIPTION OF THE DRAWINGS:

FIG. 1 is a schematic representation of an apparatus for collecting and storing a sample in accordance with an embodiment of the present invention;

FIG. 2 is a view similar to that of FIG. 1, showing an apparatus for collecting and storing a sample in accordance with another embodiment of the present invention;

FIG. 3A is a view similar to that of FIG. 1, showing an apparatus for collecting and storing a sample in accordance with yet another embodiment of the present invention;

FIG. 3B is a top plan view of a container of the apparatus shown in FIG. 3A;

FIG. 4 is a view similar to that of FIG. 1, showing an apparatus for collecting and storing a sample in accordance with a further embodiment of the present invention;

FIG. 5A-C are representations of sputum cultures using the storage composition of the present invention showing that conventionally processed sputum samples have lower yield. FIG. 5A shows same day culture after treatment in the in the media (top) using conventional methods (bottom). FIG. 5B shows sputum culture after storage in the storage media for several days in the refrigerator. FIG. 5C shows culture after sputum has been frozen in the storage media (top) or alone (bottom).

FIGS. 6A-C are representations of sputum cultures showing that conventionally processed samples may completely miss a pathogen. The pathogen is Klebsiella oxytoca. FIG. 6A shows same day cultures after treatment in the media (top) and after using conventional methods (bottom); FIG. 6B shows sputum cultures after storage in the media for several days in a refrigerator; and FIG. 6C shows cultures after sputum had been frozen in the storage media (top) or without the storage media (bottom).

FIG. 7 is a representation of a sputum cultures showing that conventionally processed samples missed S. pneumonia. FIG. 7 top culture shows that only Moroxella catarrhalis was found when sputum was stored without the storage media and FIG. 7 bottom culture shows that M. catarrhalis and S. pneumonia were isolated when sputum was mixed with the storage media.

FIG. 8 is a representation of sputum cultures showing that H. influenzae is isolated when sputum cultures were mixed with the storage media (top), but not without the storage media (bottom).

FIG. 9 is a representation of refrigerated sputum cultures when stored alone (left) or mixed with the storage media (right) showing that Pseudomonas could be isolated only in the sputum mixed with the storage media.

FIG. 10 is a representation of the effect of freezing on the yield of pathogens when the sputum was stored alone (top) or when mixed with the storage media before freezing (bottom). The pathogen is a gram negative pathogen.

FIGS. 11A-C are representations of the effect of freezing on the yield of different pathogens. All top cultures are for sputum only and the bottom cultures are for sputum cultures mixed with the storage media. FIG. 11A is for H. influenzae, 11B is for E. coli and 11C is for P. aeruginosa.

FIG. 12 is a representation of the isolation of a gram negative pulmonary pathogen sputum with the storage media. The top four plates are for sputum mixed with the storage medium and the bottom four are for the same sputum sample prior to mixing with the storage medium

FIG. 13 is a representation of isolation of Pseudomonas aeruginosa in sputum with the storage media. The top four plates are for sputum mixed with the storage medium and the bottom four are for the same sputum sample prior to mixing with the storage medium.

FIG. 14 is a representation of isolation of S. pneumoniae in sputum with the storage media. The top three plates are for sputum mixed with the storage medium and the bottom three are for sputum without the storage medium.

FIG. 15 is a demonstration of the presence of S. pneumoniae in the cultures from FIG. 14 mixed with the storage media but not without it.

### DETAILED DESCRIPTION OF THE INVENTION

The present invention provides compositions and methods for the collection and storage of clinical specimens. The specimens can be stored and transported in the compositions until analysis is carried out. The method can be used for any type of biological sample, but is particularly useful for the collection, storage and transport of sputum samples. This invention allows the microbiologist or laboratory technician to delay culture until the most opportune time without compromising yield or quality. This invention also allows an outpatient or nursing home practitioner to collect and properly process sputum samples while awaiting transport to specialty laboratories.

The storage composition of the present invention comprises a microbiological growth media; glycerol or glycerin; and a reducing agent. Optionally, non-fat dry milk can also be included.

There are several microbiological media known in the art. Some examples are Columbia broth, trypticase soy broth, Todd-Hewitt media, Mueller-Hinton broth, brain heart infusion broth and Brucella broth.

Glycerol or glycerin is used at a concentration of between about 15% to 45%.

The reducing agent can be Dithiothreitol (DTT); Dithioerythiritol (DTE); Cysteine (Cys) and Tris 2-carboxyethyphosphine (TCEP). A convenient form of DTT is sputalysin which is commercially available.

An example of a composition is as follows: Mueller-Hinton Broth: 10-50 grams; Glycerol: 60-200 ml; Dry non-fat milk: 1-5 grams; and Sputalysin: 0.4-1.0 ml of concentrated sputalysin. The corresponding amount of media is adjusted to allow the concentration of sputalysin to remain between 5-15%, preferably about 10% (i.e. 3.6-10 ml).

The present invention is particularly useful for the storage and transport of sputum samples. The present invention can be used for stool sample. In another embodiment, the present method can be used for storage and transport of samples of gastric or intestinal contents. Additionally, the present method can also be used for collection, storage and transport of surgical, wound or vaginal cultures.

Sputum can be collected in a variety of ways, including expectorating into a cup, suctioning, induction with nebulized saline and bronchoalveolar lavage through a bronchoscope. For example, sputum can be induced with hypertonic saline solution. Suitable saline concentrations are in the range of 3 to 5 percent. An example of a suitable saline concentration is 2%. This is a well accepted method of inducing sputum that contains the secretions of the lower respiratory tract. For induction of sputum, a commercially available nebulizer may be used. Generally a volume of 10-30 mls is obtained.

The present invention also provides a method for storage of biological samples. In the case of sputum, the sample is mixed with the storage composition comprising the microbiological media, glycerol and sputalysin (and optionally non-fat dry milk). Those skilled in the art will recognize that the biological samples may be contacted with the three components together or separately. The mixture is gently shaken. It is believed that by the action of the reducing agent, the disulfide bonds are broken and this contributes to reducing the viscosity of the sample. Generally 1 part sputum can be mixed with 1-50 parts storage medium. The volume of media for the sputum samples is preferably abut 0.2 to 2.0 ml of sputum in about 4-5 ml of the storage media. More preferably 1 part sputum is mixed with 3-4 parts storage medium. A similar process can be used with other types of biological samples.

In an alternate embodiment, the sputum sample can be treated first with the reducing agent. After addition of the reducing agent, the mixture is simply shaken. A reduction in the viscosity of the sputum sample can be observed. The microbiological media containing glycerol can then be added to the sputum sample treated with the reducing agent.

Following this step, the sputum sample can be transported to a clinical laboratory for analysis. It has been observed that when stored in the storage medium of the present invention, the sample is capable of supporting the pathogens for about 1-24 hours at ambient temperatures. Generally, this provides a sufficient amount of time for the samples to be transported locally to a central clinical analysis facility. The samples can also be stored in the refrigerator for up to 7 days and in the freezer for several months.

Thus, the storage media described here can be used as a) mixed with a sputum sample at the end point of collection and then processed on standard agar growth plates the same day; b) mixed with a sputum sample at the point of collection and then refrigerated for a few days prior to processing; c) mixed with a sputum sample at the point of collection and then frozen for months prior to processing; and other variations of the above. When compared to standard control, each process (same day, refrigeration and freeing) produces superior results.

In another embodiment, the samples as treated above, can be frozen for longer storage and transport. Following treatment with the microbiological media containing glycerol and sputalysin (or with sputalysin first, followed by addition of the microbiological media containing glycerol), the sample can be frozen at −20° C. or at lower temperatures. The freezing can done gradually over a period of time or can be done faster i.e., by transfer of the samples directly from the room temperature to the freezer (at −20° C. or at −70° C.).

After thawing of the samples, detection of the presence of pathogens can be carried out by routine techniques. It is expected that by using the present method, the storage and retrieval of pathogens is improved. Such pathogens include, but are not limited to, Haemophilus influenzae and Klebsiella pneumoniae, Acinetobacter baumannii, Pseudomonas aeruginosa, Stenotrophomonas maltophilia, Streptococcus pneumonia, Moraxella catarrhalis, Serratia marcescans, Pasteurella multocida, Group G streptococcus, Citrobacter freundii, Enterobacter aerogenes, Proteus mirabilis extended-spectrum beta-lactamase producing Enterobacteraciae, methicillin-resistant Staphylococcus aureus, multi-drug resistant erm/mef+Streptococcus pneumoniae, Helicobacter pylori and Mycobacterium tuberculosis as well as most gram negative and gram positive aerobic pulmonary/fecal pathogens.

Our work indicates that samples can be reliably stored at ambient temperatures for a day, or in a standard refrigerator for up to 7 days prior to culture without loss of pathogen viability. Our studies also indicate that sputum cultures can be frozen at standard freezer and dry ice temperatures (−20° C.) for up to 3 months without loss of pathogen viability. Additionally, our media works to homogenize a sample en-route to the laboratory, minimizing labor for microbiology laboratory personnel. In one study when sputum samples (50 samples) were mixed with the storage media of the present invention and frozen for 2 months, subsequent culture results were found to be identical to aliquots of the same samples processed on the collection day.

The advantages of the present invention include: 1) more reliable isolation of pathogens; 2) this invention allows centralization of laboratory staffing and supplies, which potentially eliminates the need to duplicate expensive services and supplies. In addition, fewer but more specialized laboratories may provide better service because technicians can be specialized; 3) Transport of specimens for multi-center trials and international research can be performed with certainty of results; and 4) Validation at −20° C. allows for transport of specimens in dry ice instead of the more costly, dangerous and cumbersome liquid nitrogen.

Another application stems from recognition of the importance of domestic and international collaboration in biomedical research. Current practice in collaborative research requires that each laboratory perform individual culture and isolation of pathogens prior to shipment. Due to predictable contamination with native flora, interpretation of sputum and stool cultures can be subjective. The ability to reliably allocate all culture responsibility to a central laboratory will provide welcome standardization and lower costs to multi-center trials.

In an embodiment relating to Helicobacter pylori (a documented cause of gastritis, ulcers and cancers of the stomach), the advantages include: recovery of gastric contents using a minimally invasive “string test” and immediate placement of these contents into our media will allow transport and subsequent culture of the very fastidious H. pylori organism. The string test involves the collection of gastric and intestinal contents as follows. The proximal end of a string is taped to the cheek of the subject and the remainder is swallowed and retrieved approximately 2 hours later. This procedure is standard and is well tolerated. Culture of the organism now requires invasive endoscopy and biopsy. By using the present media following the string test, analysis of samples can be performed in a less invasive fashion in an outpatient clinics and antibiotic sensitivity can be tested. It should be noted that the current methods of detecting H. pylori provide only a qualitative positive or negative result and there is no easy way to actually test the organism for antibiotic resistance. The present method would provide a convenient and less invasive method to culture the organisms so that testing for antibiotic resistance can be carried out.

In one embodiment, stool samples can also be processed using the storage medium described herein. Like sputum, in stool samples, there is a high risk of overgrowth of non-pathogenic gut flora and loss of more fastidious pathogens. Placement of stool specimens into our storage medium and device will preserve these pathogens and enable refrigeration/freezing and transport in a similar fashion as described for sputum.

Additionally, archiving of diagnostic specimens (better than archiving just the bacteria that are isolated from the original sample) for research or medico-legal use can also be done.

In one embodiment, if only increased yield of pathogen number and species is desired without the need to store or freeze the sample, it may be possible to use only sputalysin and distilled water (again, 10% concentration of sputalysin). It may also be possible to freeze the sputum in sputalysin and glycerol alone, although it is preferable to add media as well.

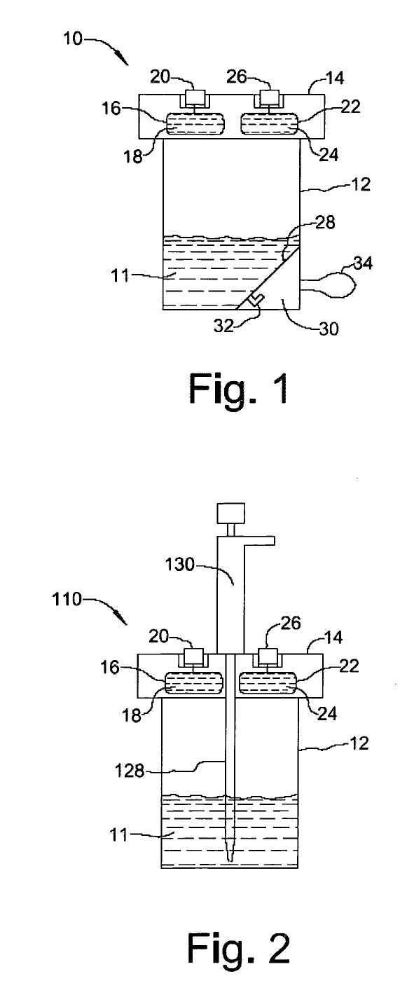

The present invention also provides an apparatus for collection and storage of a sample in a composition as described herein. As seen in FIG. 1, an embodiment of the apparatus is generally designated by reference numeral 10. Apparatus 10 comprises a container 12 for storing a sample 11 and a removable lid 14 for covering a top opening of the container. Lid 14 may be a screw top lid, a hinged lid, a snap-on lid, or any lid that seals the top opening of container 12. A screw top lid is preferred for its ease of use and manufacturing simplicity.

Lid 14 includes a first reservoir 16 for holding a first liquid 18, and a first release actuator 20 connected to first reservoir 16 such that a user may manually operate first release actuator 20 to cause release of first liquid 18 from first reservoir 16 into container 12. Lid 14 further includes a second reservoir 22 for holding a second liquid 24, and a second release actuator 26 connected to second reservoir 22 and manually operable to release second liquid 24 from second reservoir 22 into container 12. The first and second reservoirs are preferably filled with sputalysin and a microbiological growth medium, respectively. The release actuators 20 and 26 may be push-button actuators or other actuating mechanisms which open a port through the bottom of the associated reservoir or otherwise allow liquid to drain into container 12.

In the present embodiment, a protected chamber 30 is associated with container 12 by providing a sealed partition 28 at a bottom portion of container 12, whereby some of sample 11 can be stored in pure form. A one-way valve 32 permits flow into protected chamber 30 from the remaining portion of container 12. A suction device 34 arranged to communicate with protected chamber 30 is operable to create a pressure differential between protected chamber 30 and the remaining portion of container 12 to thereby draw liquid into protected chamber 30. Suction device 34 may be a balloon suction device, piston and cylinder, or other suction device capable of creating a pressure differential.

Apparatus 10 works as follows:
1. Open lid 14.
2. Expectorate sputum or other diagnostic sample (stool) into container 12 and replace lid 14.
3. Use suction device 34 to suck a portion of the sputum sample into protected chamber 30 through one-way valve 32 (this portion of sample will be used for gram-stain).
4. Operate release actuators 20 and 26 to release sputalysin and medium into container 12. Manually agitate apparatus 10.
5. In the laboratory, open lid 14 and pipette sample and media onto plates for detection.

Another embodiment of the apparatus is shown in FIG. 2 and identified by reference numeral 110. Apparatus 110 is similar to apparatus 10 described above, except a protected chamber 130 is defined by a pipette 128 arranged to draw a portion of sample 11 from container 12 prior to operation of release actuators 20 and 26. Consequently, partition 28, valve 32, and suction device 34 are eliminated.

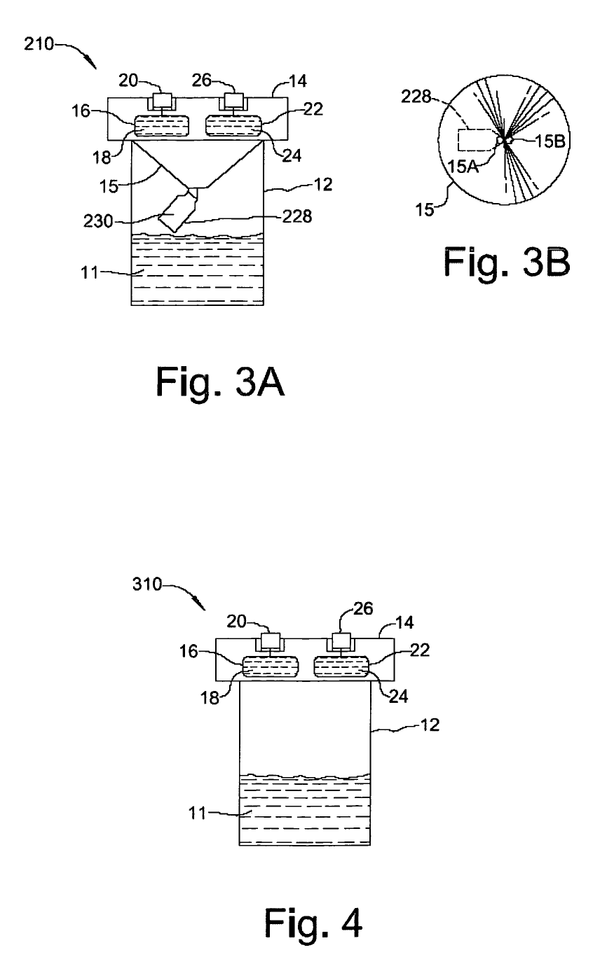

Yet another embodiment of the apparatus is shown in FIGS. 3A and 3B and designated by reference numeral 210. In this embodiment, container 12 includes a funnel portion 15 near the container opening, and a protected chamber 230 is defined by an auxiliary vessel 228 depending from funnel portion 15, wherein funnel portion 15 channels sample by gravity through orifice 15A to protected chamber 230 and through orifice 15B to the remaining portion of container 12. Orifice 15A is closed off by a suitable mechanism (not shown) prior to operation of release actuators 20 and 26.

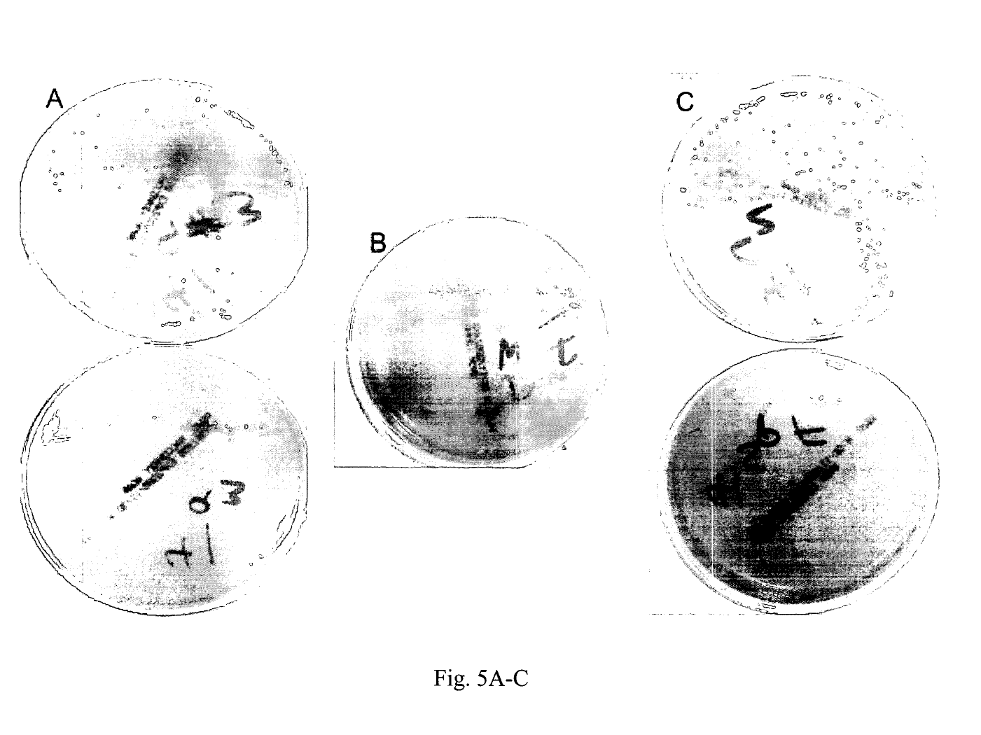

FIG. 4 shows an apparatus 310 according to a further embodiment wherein no gram-stain is required or desired, and thus there is no need to isolate a portion of the original sample. Apparatus 310 is similar to apparatus 10 of shown in FIG. 1, except that partition 28, valve 32, and suction device 34 are eliminated such that no protected chamber 30 is defined.

The following examples are presented to further illustrate the invention.

### EXAMPLE 1

17.4 grams of Mueller-Hinton Broth
2.5 grams of dry non-fat milk
120 ml of glycerol

The three ingredients listed above were mixed with distilled water to make a volume of 400 ml and autoclaved. When it was ready to be mixed with a sputum sample, 3.6 ml of the above were mixed with 0.4 ml of concentrated dithiothreitol in a phosphate buffer (sputalysin). The 4.0 ml total was mixed with the sputum sample and the sample is homogenized for about 30-60 minutes. Fifty microliters are then placed on agar media plates and set up for semi-quantitative culture by standard methods.

### EXAMPLE 2

This examples demonstrates that using the storage media of the present invention, pathogenic bacteria can be recovered after freezing and that storage at −20° C. is sufficient for recovery of the pathogens. For the experiments, expectorant sputum was collection and mixed with one part 2×Mueller-Hinton broth+30% glycerol. Three separate aliquots of the mixture were processed for storage at −20° C.; storage at −70° C. and −20° C.×14 days was followed by transfer to −70° C. respectively. Samples are thawed after 1-16 weeks and culture results are compared to initial findings. Over 50 isolates were examined and we have observed a >95% concordance rate with a variety of pathogenic gram-negative and gram-positive organisms including H. influenzae, S. aureus, P. aeruginosa, and M. catarrhalis. No significant difference was observed between storage at −20° C. and −70° C. This indicates samples can safely be transported at −20° C.

### EXAMPLE 3

This examples demonstrates that various pathogens can be recovered after refrigeration or freezing. Approximately 20 samples have been evaluated. One-hundred percent of pathogens were recovered after both freezing and refrigerating the samples. The quantitative yield has been significantly greater in each specimen. Some examples are presented in FIGS. 5-15.

FIG. 5 is a sputum sample growing Pseudomonas aeruginosa and Serratia macresens. Column A illustrates the greater number of colonies achieved with the media, and shows that it is possible to distinguish the two organisms on the plate that was streaked with the sputum+media mix. The lower (standard) plate does not allow this distinction (i.e. a significant respiratory pathogen would have been missed in this patient. Column B illustrates that it is possible to replicate results after storage in the refrigerator. Column C illustrates that frozen samples produce results that are indistinguishable from the original same day sample, despite freezing.

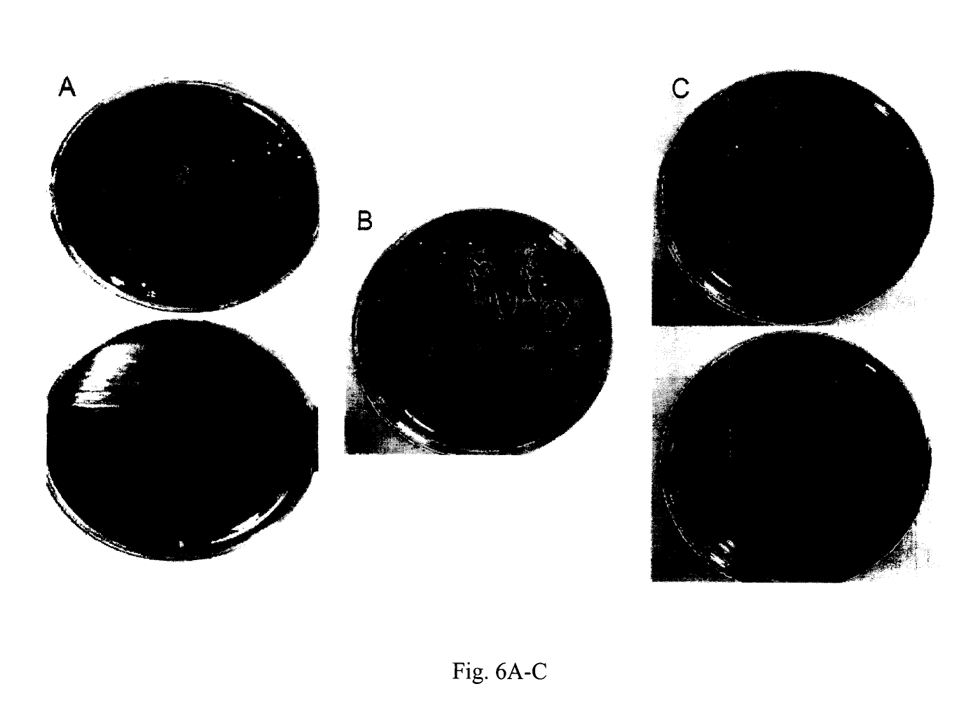

FIG. 6 is the same as exhibit A, but with different organism (Klebsiella oxytoca).

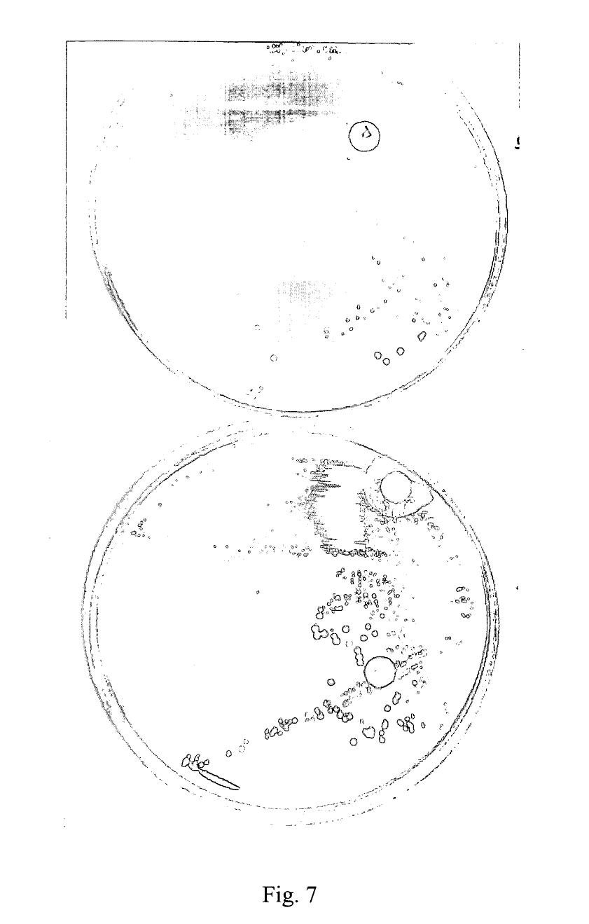

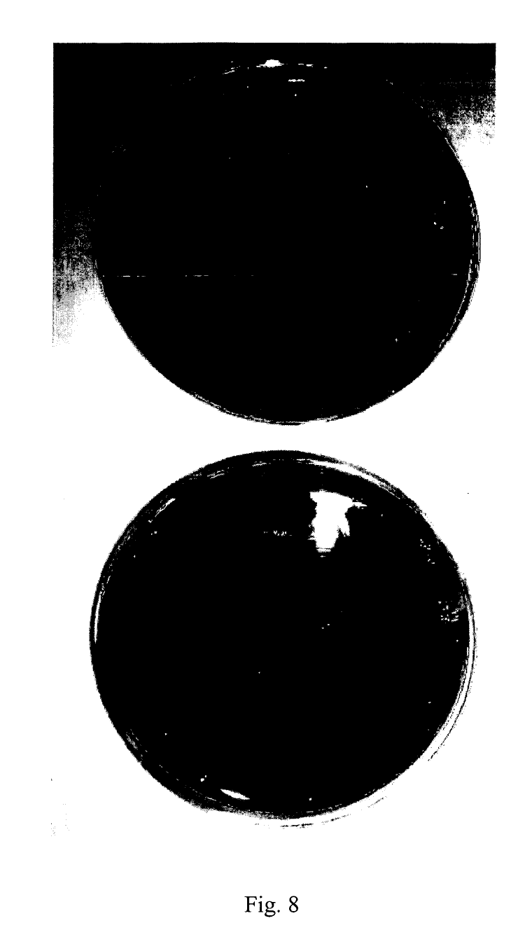

FIG. 7 is same day processing. The lower plate was streaked with media plus sputum mix, while the upper plate was just streaked with sputum. Two significant respiratory pathogens (Streptococcus pneumoniae and Moraxella catarrhalis) were isolated on the lower plate, while only M catarrhalis was isolated on the upper plate).

FIG. 8 is same day processing. Haeomophilus influenzae was isolated from media plus sputum mix, while no organism was isolated from the standard sputum.

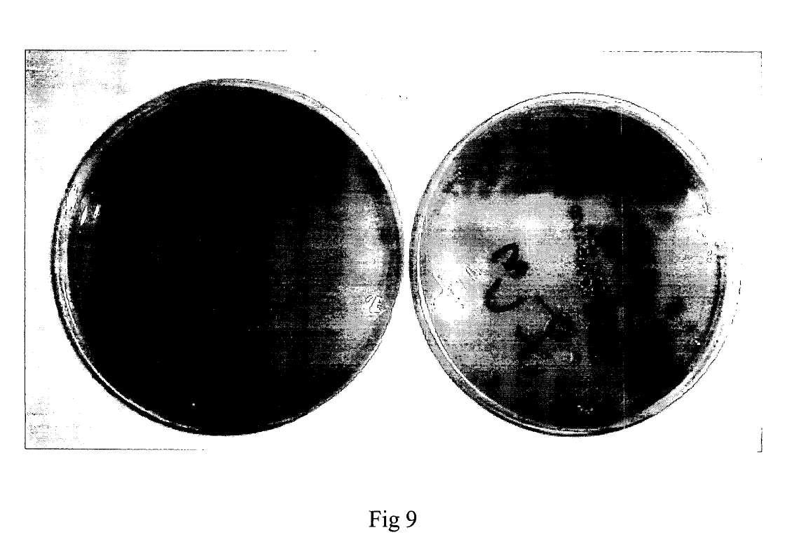

FIG. 9 is refrigerated samples. Pseudomonas aeruginosa was isolated from the media plus sputum mix only. Refrigerated sputum (alone) lost the pathogen.

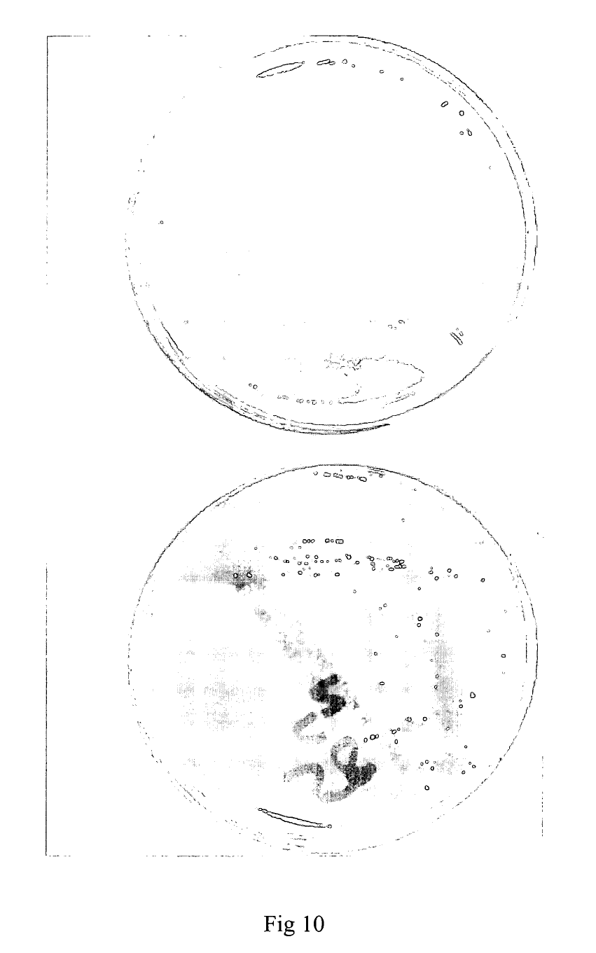

FIG. 10 is frozen samples. A Gram-negative pathogen was isolated from sputum plus media mix, but not from frozen sputum.

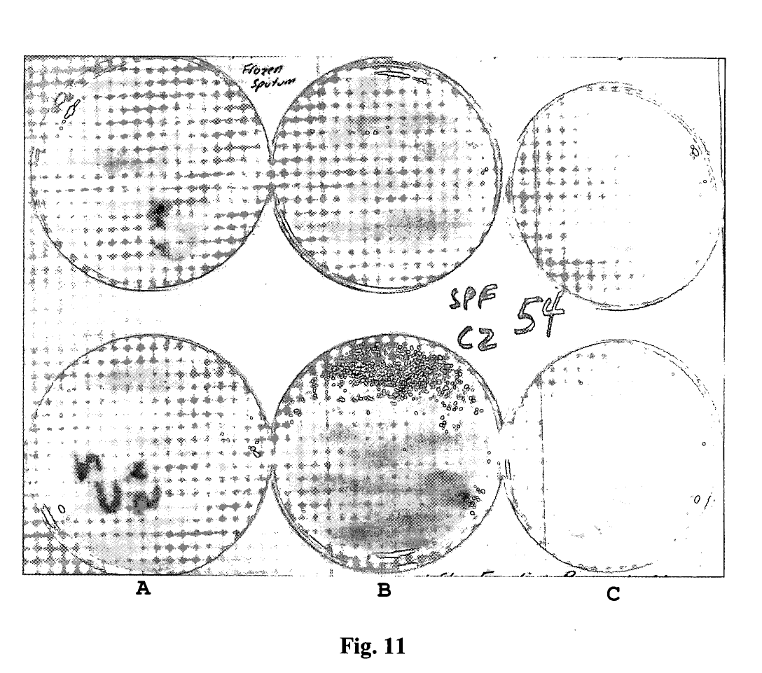

FIG. 11 are frozen samples. The bottom row shows agar plates that were streaked with a media plus sputum mix that was frozen for several days. Three pathogens were isolated (H. influenzae, E. coli and P. aeruginosa). In the frozen sputum, only one pathogen was isolated (H. influenzae).

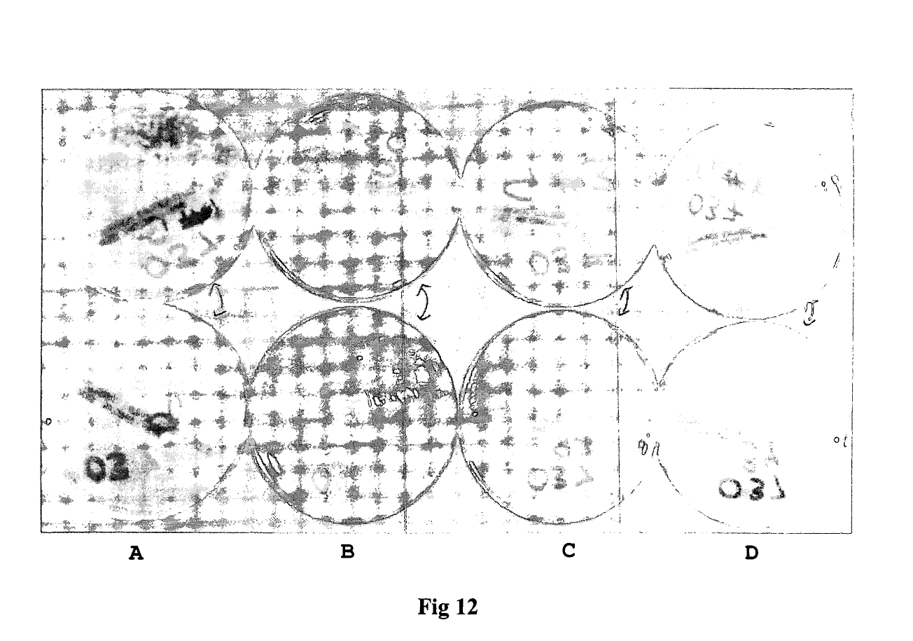

FIG. 12 are frozen samples. This illustration demonstrates that these findings are reliably reproducible. Each plate on the top and bottom row comes from one of 4 separate streaking trials. For the bottom plates, the loop was inserted into the sputum at four different locations and then each one streaked on a plate. Then the sputum sample was treated with the storage medium, frozen, thawed and then again loop samples from four different location were streaked on the four separate plates shown on top. In each case, the sputum plus media that was frozen produces a reliable and consistent number of organisms, whereas the standard sputum either produced no or few organisms providing inconsistent results. The pathogen is a gram negative pathogen.

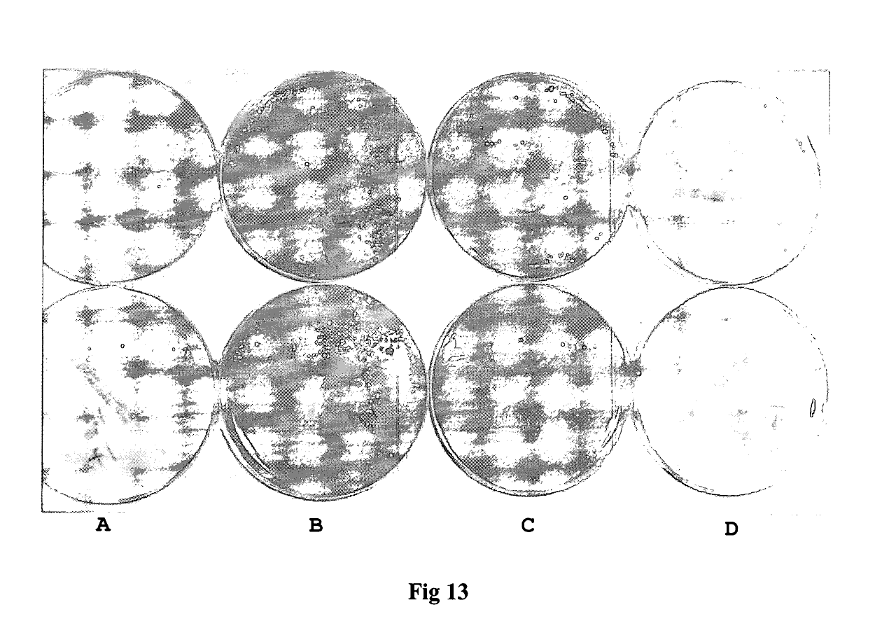

FIG. 13 are sputum samples processed as in FIG. 12 for Pseudomonas aeruginosa.

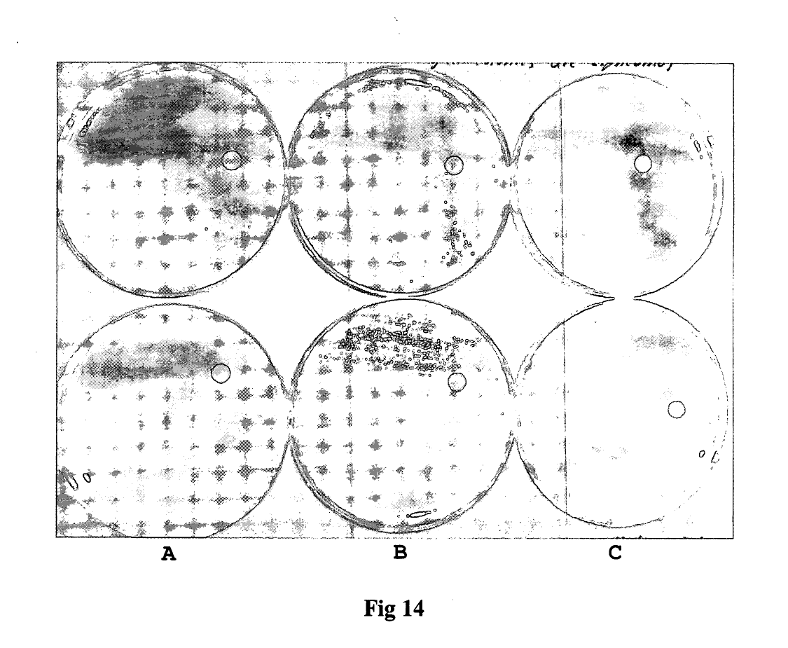

FIG. 14 is same day prep. The top row is composed of media plus sputum plates. The S. pneumoniae present in the sample is easily distinguished from background mouth flora colonies that produce a similar appearance (and therefore lead to a frequent “normal flora” results when there is actually a pathogen present). The bottom row demonstrates that it is difficult to find the pathogen in the standard methods.

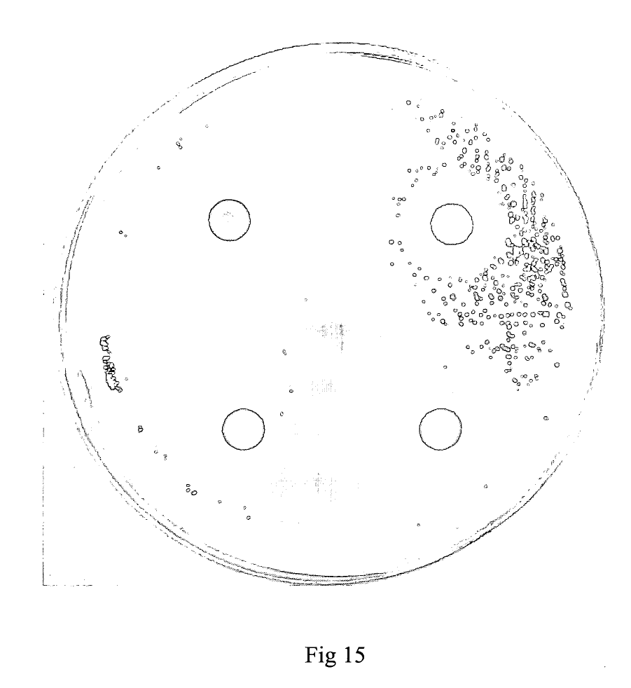

FIG. 15 is individual colonies isolated from the media plus sputum plates seen in FIG. 14. The S. pneumoniae demonstrates sensitivity to the optichin (P disc) and exhibits a zone of inhibited grown around the disc. Standard oral “normal flora” looks similar to the S. pneumoniae colonies, but does not demonstrate the inhibited growth around the disc. The pathogen could only be readily isolated from the media plus sputum plate.

### EXAMPLE 4

An additional study to evaluate the utility of our storage media to collect and transport H. pylori using the string test technique was performed in Lima, Peru in June 2005. Although the sample size was small, viable organisms were successfully recovered using the storage composition of the present invention.

### EXAMPLE 5

This example is another demonstration of the effectiveness of the stprage media in identifying bacterial pathogens in sputum and in the isolation the bacterial pathogens in culture from sputum samples that were frozen after collection. Expectorated sputum samples that were submitted for routine culture to the clinical microbiology laboratory at the Buffalo VA Medical Center were studied. After obtaining approval from the Institutional Review Board, we obtained the remainder of the sputum samples after laboratory personnel had completed their studies.

The effectiveness of the transport media in identifying bacterial pathogens was compared to routine methods used in most clinical microbiology laboratories (inserting an inoculating loop into the sputum sample to obtain a portion of the sample and inoculate the sample onto agar plates). Sputum sample were suspended in an equal volume of storage media of the present invention and mixing the sample for 15 minutes. An aliquot of this sample was inoculated onto agar plates for culture. Thirty seven sputum sample were studied in this way. Using the standard method, a total of 35 potential pulmonary pathogens were isolated from the 37 sputum samples. A total of 42 potential pulmonary pathogen were isolated from the same samples with the use of the transport media. The bacteria present in the sputum samples were Streptococcus pneumoniae, Haemophilus influenzae, Moraxella catarrhalis, Klebsiella pneumoniaem, Pseudomonas aeruginosa, Serratia marcescans, Staphylococcus aureus, Pasteurella multocida, Group G streptococcus, Citrobacter freundii, Enterobacter aerogenes, Stenotrophomonas maltophilia and Proteus mirabilis. The bacteria that were detected by using the storage media but were missed by the standard loop culture without the storage media were Serratia marcescans, Haemophilus influenzae, Proteus mirabilis, Klebsiella pneumoniae, Enterobacter aerogenes, and Stenotrophomonas maltophilia. While not intending to be bound by any particular theory, it is believed that suspension of the sputum sample in the media which preserves the viability of the bacteria in the sample accounts for the improvement in the result compared to the routine method which is prone to sampling error because only a small portion of the sputum specimen is sampled by the inoculating loop.

The effectiveness of the method in allowing for the freezing of sputum samples and recovery of bacterial pathogens at a later time was also studied in this example. After an aliquot of sputum sample suspended in transport media was removed for culture as described in the previous paragraph, the sample was frozen at −20° C. As a control, an aliquot of the sputum sample that was not suspended in transport media was also frozen. One to 3 days later, the samples were thawed, aliquots were inoculated onto appropriate agar plates and bacterial pathogens were identified. A total of 40 of the 42 pathogens identified with immediate culture were recovered from the frozen samples. Only 18 pathogens were identified in the same sputum samples frozen without transport media. Freezing in transport media allowed culture and identification of more pathogens compared to routine methods used in clinical microbiology laboratories which identified 35 pathogens from the same samples. Bacteria which were detected using the storage medium of the present invention but were missed when the storage medium was not used were: Streptococcus pneumoniae, Haemophilus influenzae, Klebsiella pneumoniae, Pseudomonas aeruginosa, Serratia marcescans, Staphylococcus aureus, Citrobacter freundii, Enterobacter aerogenes, and Stenotrophomonas maltophilia

The invention has been described through specific embodiments. However, routine modifications to the compositions, methods and devices will be apparent to those skilled in the art and such modifications are intended to be covered within the scope of the invention.

### REFERENCES

1. Yourassowsky E. Collection and transport of specimens for bacteriological analysis: A neglected subject in medical teaching. Infection 1980;8:S143-s145.
2. Tilton R C, Maderaza E, Iannini P, Quintiliani R. The bacteriological examination of sputum. Ann Clin Lab Sce 1974;4:60-3.
3. Jefferson H, Dalton H P, Escobar M R, Allison M J. Transporation delay and the microbiological quality of clinical specimens. Am J Clin Pathol 1975;64:689-93.
4. May J R, Delves D M. The survival of Haemophilus influenzae and pneumococci in specimens of sputum sent to the laboratory by post. J Clin Path 1964:17:254-256.
5. Farrell D J, Jenkins S G, Brown S D, Patel M, Lavin B S, Klugman K P. Emergence and spread of Streptococcus pneumoniae with erm(B) and mef(A) resistance. Emerg Infect Dis. 2005 June;11(6):851-8.
6. Bartlett J G, Dowell S F, Mandell L A, File Jr T M, Musher D M, Fine M J. Practice guidelines for the management of community-acquired pneumonia in adults. Infectious Diseases Society of America. Clin Infect Dis. 2000 August;31(2):347-82. Epub 2000 Sep. 7.
7. Paradisi F, Corti G. Antibiotic resistance in community-acquired pulmonary pathogens. Semin Respir Crit Care Med. 2000;21(1):33-44.
8. del Campo R, Morosini M I, de la Pedrosa E G, Fenoll A, Munoz-Almagro C, Maiz L, Baquero F, Canton R; Spanish Pneumococcal Infection Study Network. Population structure, antimicrobial resistance, and mutation frequencies of Streptococcus pneumoniae isolates from cystic fibrosis patients. J Clin Microbiol. 2005 May;43(5):2207-14.
9. Madison J M, Irwin R S. Expectorated sputum for community-acquired pneumonia. Arch Intern Med 2004;164:1725-1727.
¹May J. The bacteriology of chronic bronchitis. Lancet 1953:265;534-7.
10. Guidelines for the manangement of adults with hospital acquired, ventilator associated, and health-care associated pneumonia. Am J Respir Crit Care Med 2005;171:388-416.

1. A method for storing and transporting sputum comprising the steps of:
a) collecting expectorant sputum from an individual;
b) treating the expectorant sputum with a reducing agent
c) mixing the sample from b) with a composition comprising a microbiological growth medium and 15-45% glycerol;
d) storing the sample from c) at room temperature, refrigerated temperature or freezer temperature;
e) transporting the sample from d) to a facility for further storage and/or culture analysis.
wherein the yield of pathogens is comparable or better than the yield from a freshly cultured sputum sample.

2. The method of claim 1, wherein steps b) and c) are performed simultaneously in the same container.

3. The method of claim 1, wherein the pathogen is a gram negative pathogen or a gram positive aerobic pulmonary or fecal pathogen.

4. The method of claim 1, wherein the pathogens are selected from the group consisting of Haemophilus influenzae, Klebsiella pneumoniae, Acinetobacter baumannii, Pseudomonas aeruginosa, Stenotrophomonas maltophilia, Streptococcus pneumonia, Moraxella catarrhalis, Serratia marcescans, Pasteurella multocida, Group G streptococcus, Citrobacter freundii, Enterobacter aerogenes, Proteus mirabilis, extended-spectrum beta-lactamase producing Enterobacteraciae, methicillin-resistant Staphylococcus aureus, multi-drug resistant erm/mef+Streptococcus pneumoniae, Helicobacter pylori and Mycobacterium tuberculosi.

5. A composition for storage and transport of biological samples according to claim 1 comprising a microbiological growth medium; 15-45% glycerol, and a reducing agent.

6. The composition of claim 5, wherein the microbiological growth medium is selected from the group consisting of Columbia broth, trypticase soy broth, Todd-Hewitt media, Mueller-Hinton broth, brain heart infusion broth and Brucella broth.

7. The composition of claim 5, wherein the microbiological growth medium is Mueller-Hinton broth.

8. The composition of claim 6, wherein the reducing agent is selected from the group consisting of Dithiothreitol (DTT); Dithioerythiritol (DTE); Cysteine (Cys) and Tris 2-carboxyethyphosphine (TCEP).

9. The composition of claim 8, wherein the reducing agent is DTT.

10. The composition of claim 5, wherein the microbiological growth medium is Mueller-Hinton broth, and DTT is present at a concentration of about 10%

11. The composition of claim 5, wherein the biological sample is sputum.

12. The composition of claim 5, wherein the biological sample is stool.

13. An apparatus for collecting and storing a sample to be tested, the apparatus comprising:
a container having an opening through which a sample is collected; and
a removable lid for selectively covering the opening, the lid including:
a first reservoir for holding a first liquid;
a first release actuator connected to the first reservoir, the first release actuator being manually operable to release the first liquid from the first reservoir into the container;
a second reservoir for holding a second liquid; and
a second release actuator connected to the second reservoir, the second release actuator being manually operable to release the second liquid from the second reservoir into the container.

14. The apparatus according to claim 13, further comprising a protected chamber associated with the container for receiving a portion of a collected sample to be isolated from liquid held in the first and second reservoirs.

15. The apparatus according to claim 14, wherein the protected chamber is provided at a bottom portion of the container and communicates with a remaining portion of the container through a one-way valve.

16. The apparatus according to claim 15, further comprising a suction device communicating with the protected chamber, the suction device being operable to create a pressure differential between the protected chamber and the remaining portion of the container.

17. The apparatus according to claim 14, wherein the protected chamber is defined by a pipette arranged to selectively draw a portion of a collected sample from the container.

18. The apparatus according to claim 14, wherein the container includes a funnel portion near the container opening, and the protected chamber is defined by an auxiliary vessel depending from the funnel portion, wherein the funnel portion channels sample to both the protected chamber and a remaining portion of the container by gravity.

19. The apparatus according to claim 13, further comprising sputalysin held in the first reservoir.

20. The apparatus according to claim 19, further comprising a growth medium held in the second reservoir.
